(19) United States
(12) Patent Application Publication
(10) Pub. No.: US 2006/0051355 A1
(43) Pub. Date: Mar. 9, 2006

(54) Methods and means for the treatment of immune-related diseases

(76) Inventors: Ypke V.J.M. van Oosterhout, Nijmegen (NL); Janny E. van Emst, Wijchen (NL)

Correspondence Address: TRASK BRITT, P.O. BOX 2550, SALT LAKE CITY, UT, 84110, US

(21) Appl. No.: 11/069,823

(22) Filed: Mar. 1, 2005

Related U.S. Application Data
(63) Continuation of application No. 09/668,555, filed on Sep. 22, 2000.
Application No. 09/668,555 is a continuation of application No. PCT/NL99/00156, filed on Mar. 19, 1999.
(60) Provisional application No. 60/079,086, filed on Mar. 23, 1998.

(30) Foreign Application Priority Data
Mar. 23, 1998 (EP) 98200917.7

(51) Int. Cl. A61K 39/395; A61K 36/185

(52) U.S. Cl. 424/183.1; 424/731

(57) ABSTRACT

The present invention provides novel means and methods for treating unwanted side effects in transplantations, such as GVHD and allograft rejection. The invention provides immunotoxins comprising an antibody and a toxic substance, whereby cocktails of such conjugates are directed to different targets associated with one population of cells, wherein one target is chosen from CD3 or CD7. The preferred combination is a cocktail directed against both CD3 and CD7.

### CROSS-REFERENCE TO RELATED APPLICATIONS

This application is a continuation of U.S. application Ser. No. 09/668,555, filed 22 Sep. 2000, which is a continuation of International Application No. PCT/NL99/00156, filed on 19 Mar. 1999 designating the United States of America and published in English as PCT International Application No. WO 99/48534, which itself claims priority from U.S. Provisional Patent Application Ser. No. 60/079,086, filed 23 Mar. 1998, the entirety of all of which are incorporated by this reference.

### TECHNICAL FIELD

The invention relates to the field of biotechnology, specifically to immune system related diseases, and in particular, to novel means and methods for treating these diseases. More particularly, the invention provides novel means for eliminating or suppressing populations of unwanted CD3 and/or CD7 positive cells. Typically, the invention finds applications in the field of allogeneic bone marrow transplantation.

### BACKGROUND

Allogeneic bone marrow transplantation (BMT) is a world-wide accepted method of treating a number of severe disorders like leukemia, myelo-dysplastic syndrome, bone marrow failure, immune deficiency, storage diseases and hemoglobinopathies (1-6). For a good engraftment, the bone marrow must contain a minimum number of T cells (7-10). These cells may also confer benefit as they contribute to the so-called graft-versus-leukemia effect which involves the elimination of residual malignant cells (11-13). However, donor T cells may react with normal tissues of the host causing graft-versus-host-disease (GVHD) which results in serious damage to the skin, liver and gastrointestinal tract (2, 14-16). When this disease occurs within the first three months after BMT, it is classified as acute GVHD. When GVHD develops at a later stage, it is referred to as chronic GVHD. The severity of the clinical symptoms is expressed in four grades, grade I refers to minimal GVHD, and grade IV refers to the most severe form. Grade IV GVHD is usually fatal and involves epidermiolysis, liver failure, and severe diarrhea (14).

The incidence and severity of GVHD can be diminished by depleting T cells from the graft. At the University Hospital Nijmegen, 98% of lymphocytes are depleted from the graft using counterflow centrifugation (17). However, despite depletion of the vast majority of T cells, GVHD can still occur. The annual incidence of grade II through IV GVHD at the University Hospital Nijmegen is approximately six out of a total of forty allogeneic bone marrow transplant recipients. Worldwide, GVHD occurs in 30-70% of HLA-matched recipients and contributes to death in 20-40% of those affected (2, 18). Even if a patient survives severe GVHD, the disease results in long-lasting disability and morbidity leading to repeated admission to the hospital. The development of an effective alternative treatment for GVHD will, therefore, have a major impact on both survival and the quality of life of allogeneic BMT recipients.

Current treatment is usually as follows. An immuno-prophylaxis cyclosporin is administered intravenously from one day before transplantation onwards (3 mg/kg/d for 15 days, and thereafter 2 mg/kg/d). As soon as the patient can take oral medication, cyclosporin is given orally (6 mg/kg/d). If the patient develops GVHD, first line therapy in the form of corticosteroids is given (prednisone: 1 mg/kg/d). In the event the patient does not respond to this therapy within 48 hours, or if the GVHD is progressive within 24 hours, high-dose methylprednisolone (Solumedrol 4×250 mg/day) is given as second line therapy. When high-dose methylprednisolone fails, patients are currently treated with Leukotac (a-IL2-receptor MoAb) (19). Although responses are seen in some cases during administration of Leukotac, GVHD normally recurs once treatment is discontinued. GVHD will eventually be fatal for about 90% of the patients who do not respond to the second line therapy within 3-5 days.

### SUMMARY OF THE INVENTION

The present invention provides novel means and methods for, among other things, the treatment of GVHD using molecules specifically recognizing receptors on cells to be eliminated. In a number of preferred embodiments, at least one such molecule will be coupled to a toxic moiety. Such molecules are often referred to as immunotoxins.

An immunotoxin (IT) is a conjugate of, for instance, a monoclonal antibody (MoAb), usually developed in a mouse, coupled to a potent toxin such as the A-chain of the potent plant toxin ricin (RTA) (FIG. 1).

The mechanism of action of the IT is depicted in FIG. 2. The MoAb-moiety first specifically binds to an antigen expressed on those cells which are to be eliminated, and, subsequently, the entire antigen-IT complex is internalized. Once inside the cell, the bond between the MoAb and the toxin is broken thereby releasing free toxin into the cytoplasm. The released toxin irreversibly inhibits protein synthesis by means of a catalytic reaction which culminates in the cell's death. Since the toxin-moiety is incapable of entering the cell autonomously and is inactive outside the cell, ITs are only hazardous to cells that express the specific target antigen and are capable of internalizing the IT complex.

Since their introduction in 1979, ITs have been used to treat a variety of diseases including solid and diffuse tumors, immunological disorders and viral infections. ITs have been investigated at the Department of Hematology of the University Hospital Nijmegen since 1984 within the framework of bone marrow transplantation to eliminate malignant T/B cells from autologous bone marrow grafts and normal T cells from allogeneic bone marrow grafts (21-31).

Clinical results published so far are mainly from Phase I/II studies. The commonly observed Phase I dose-limiting toxicities of RTA-ITs are vascular leak syndrome (VLS) and myalgias. These side effects, due to the “bystander” effects of the toxin, appeared to be transient and generally ceased shortly after immunotoxin administration was discontinued (32). Two different trials witnessed toxicity due to cross-reactivity of the MoAb-part with undesired tissue (33, 34). In both cases the IT, directed against ovarian carcinoma and breast carcinoma, respectively, cross-reacted with neural tissue resulting in severe neurotoxicity. These observations stress the importance of extensive preclinical screening for cross-reactivity with normal tissue. A common feature in most clinical trials was the development of human antibodies against both the MoAb and the toxin part of the IT (32). These humoral immune responses were not associated with enhanced toxicity nor allergic reactions, but decreased the serum half-life of the IT. Nevertheless, clinical responses were seen even in the face of anti-IT antibody (35-37).

The efficacy of treatment with ITs, as observed in the Phase I trials, is strongly influenced by the accessibility of target cells. So far, treating solid tumors has met with little success, whereas treatment of disorders like lymphomas and certain immunological diseases resulted in partial and complete responses in 12-75% of cases (38). These results are impressive considering that treatment of cancer with more than 90% of the drugs currently available produced fewer than 5% of the partial and complete responses in Phase I trials (38).

Thus, the present invention provides a pharmaceutical composition for eliminating or reducing the number of unwanted CD3 and/or CD7 positive cells, comprising a mixture of at least a first molecule specifically recognizing CD3 or CD7 and at least a second molecule specifically recognizing another ligand receptor associated with the surface of such an unwanted cell, whereby at least one of the specifically recognizing molecules is provided with a toxic moiety. A pharmaceutical composition is defined herein as any composition which can be administered to an individual, be it as one single dose or as a regimen of doses, by any viable route, preferably by intravenous administration, optionally containing usual vehicles for administration and/or components of regular treatment of the relevant immuno-related disease. Unwanted cells are any cells that comprise CD3 and/or CD7 (and, of course, many other) molecules associated with the cell surface, which cells are involved in a pathological condition in an individual. Typically these cells are T cells, NK cells, or other cells playing a role in GVHD or allograft rejection. Also, aberrant cells (T cell leukemias or lymphomas, for instance) comprising CD3 and/or CD7 (preferably both) can be eliminated or suppressed according to the invention.

A “molecule specifically recognizing CD3 or CD7 or another ligand-receptor” is a term well understood in the art and means any molecule having a relatively high binding affinity and specificity for CD3, CD7, or the receptor. Typically, the molecule may be a ligand for a receptor or an antibody for either CD3, CD7, or another receptor, which antibody may be truncated, humanized, or altered in any other way without losing its specificity (such alterations are herein defined as derivatives and/or fragments). “Receptor” is defined as any molecule capable of a specific interaction. Toxic moieties are basically any and all molecules that lead to toxicity for the target cell either directly or indirectly, including but not limited to lectins, ricin, abrin, PE toxin, diphtheria toxin, radio-isotopes, cytostatic drugs such as adriamycin, apoptosis-inducing agents and prodrug converting substances together with prodrugs such as the thymidin kinase and gancyclovir combination. Ricin A is preferred. In order to prevent the binding of ricin A to carbohydrate-receptors expressed by liver cells, deglycosylated ricin A (dgRTA) (20) is preferred. In prodrug embodiments, typically one of the molecules specifically recognizing CD3, CD7, or a receptor may be provided with the converting agent and the other with the prodrug. CD3, however, does not need to be coupled to a toxic moiety to have an effect, since it blocks the interaction of the T cell receptor with APCs. This is one of the advantages of the present invention which is not present in the prior art.

As stated above, it is preferred that both CD3 and CD7 are targeted by specific binding molecules therefor. This cocktail has proven itself in preliminary clinical studies in an embodiment where the toxic moiety is attached to both specifically binding antibody molecules. A surprising effect of this cocktail is that although in some cases GVHD relapse occurs, unlike current treatments this relapse is now treatable with low dose corticosteroids. Thus, such a combination is another preferred embodiment of the present invention. The invention also provides a pharmaceutical composition as described above, whereby said first molecule specifically recognizes CD3 and said second molecule specifically recognizes CD7.

The toxic moiety may be coupled to the specifically binding molecule in any manner. For example, the coupling can occur with a fusion protein by recombinant means, typically including a protease cutting site between a binding molecule and a toxic (protein) moiety, but for ease of manufacturing and freedom of choice in toxic moieties, chemical coupling is preferred, optionally by an acid-labile linker. Upon internalization, a conjugate typically goes through a lysosome.

The invention also provides a pharmaceutical composition wherein at least two molecules specifically recognizing different receptors are provided with toxic moieties, which may be the same or different toxic moieties. A major advantage to using different toxic moieties when the side effects of the moieties are different, is that higher doses can be given. Typically the pharmaceutical compositions according to the invention may further comprise at least one further molecule specifically recognizing CD5, CD2, CD4, CD8, or an IL-2 receptor, which may also be coupled to a toxic moiety. This may provide higher efficacy, but may also be used to provide higher specificity for groups of cells or in combination with, for instance, prodrug regimes.

It is preferred that when the molecule specifically recognizing CD3 is an antibody, that the antibody is of the IgG gamma-2B class, because this antibody does not fix human complement or bind human Fc-receptors and, thereby, does not induce cytokine release by the targeted T cells. Thus, the invention, in yet another embodiment, provides a pharmaceutical composition which recognizes CD3 wherein the first molecule is a gamma2B IgG antibody or a derivative thereof.

Doses used are given in the detailed description below. The limits of doses of immunotoxins in regimens such as provided herein are typically dependent on the immunotoxin, both because of the specificity and affinity of the specific binding molecules as well as because of the different tolerated doses for different drugs. Expressed in equivalents of deglycosylated ricin A, the limits will be generally within at least 25 micrograms per square meter body surface (a grown human of 80 kg, typically has 2 square meters of surface), preferably 100 micrograms of ricin A per square meter of body surface. This is a lower limit of what may be given in one or more doses over one or more days of treatment. Generally the total doses of ricin A equivalents should not be higher than 25 mg per square meter body surface.

Typically the compositions according to the invention will be used for the treatment of GVHD, graft rejections, T cell leukemias, T cell lymphomas or other CD3 and/or CD7 positive malignancies, autoimmune diseases, or infectious immune diseases such as HIV-infection. As stated above, in GVHD and graft rejection, a typical effect is seen in that after treatment with a composition as disclosed herein, the relapse is treatable with low doses of corticosteroids. Such a regimen is also part of the invention.

Thus, the invention also provides a kit of pharmaceutical compositions for treating GVHD and/or graft rejection comprising a pharmaceutical composition comprising at least one corticosteroid. The invention leads to a drop in numbers in the population of unwanted cells to at least 20% of the original amount, usually even to 5% or less. In contrast to what prior art regimes have accomplished, typically, this number stays low over a prolonged period of time. Another advantage of the present invention is that the exemplified composition not only targets T cells, but also NK cells.

### BRIEF DESCRIPTION OF THE DRAWINGS

FIG. 1 is a view of an example of an immunotoxin.

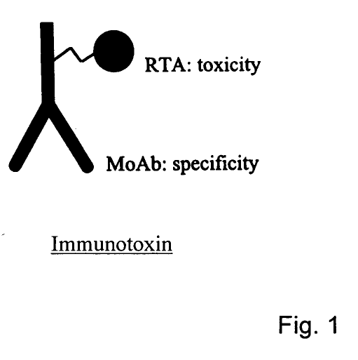

FIG. 2 is a view of an example of the mechanism of action of an immunotoxin with an antigen.

FIG. 3 is a view of a SMPT.

FIG. 4 is a bar graph depicting the proliferation of responder cells when SPV-T3a was added following the initiation of a mixed lymphocyte culture. The graph illustrates that alloactivation was blocked when SPV-T3a was added following the initiation of the mixed lymphocyte culture.

FIG. 5 consists of two graphs charting incubation of a CTL-clone treated with SPV-T3a as compared to a control group and IgG2b-mAb. The first graph illustrates the reduction of CTL-cytotoxicity directly following treatment. The second graph shows the reduction of the CTL-cytotoxicity on day 5.

FIG. 6 is a graph illustrating the reduction of NK-activity determined by a ⁵¹Cr labeled release incubating with saturating amounts of WT1-dgRTA, IgG2a-dgRTA, WT1 and SPV-T3a-dgRTA.

FIG. 7 consists of a chart and graph illustrating the weight changes following the administration of the IT-cocktail.

FIG. 8 consists of two graphs comparing SPV-T3a-dgRTA, WT1-dgRTA, and IT cocktail at different serum concentrations. Each graph illustrates the rise in serum concentration following infusion.

FIG. 9 consists of two graphs illustrating CK levels after infusion of the IT cocktail. The arrows pointing to the graphs show the times that the IT cocktail was placed in the system. The graphs illustrate a rise of CK-levels following infusion.

FIG. 10 is a graph illustrating the reduction of circulating T cells and NK cells following a first infusion. The graph is based upon a 24 hour scale.

FIG. 11 consists of two pictures of skin biopsies of a patient. Picture A depicts the epidermis before the IT-cocktail treatment while picture B depicts the epidermis two weeks after the IT-cocktail treatment. Picture A is typical for severe GVHD.

FIG. 12 is a graph depicting the response of lymphocytes in a test subject after the IT cocktail was placed in the system.

### DETAILED DESCRIPTION OF THE INVENTION

The rationale for ITs to treat GVHD is that these conjugates can be used for an efficient and specific eradication of immunocompetent T cells responsible for the disease. In this perspective, ITs might be more effective and may cause less side effects than broadly immunosuppressive reagents such as cyclosporine and corticosteroids. In 1990, Byers et al. reported a Phase I clinical trial in which they intravenously administered an anti-CD5 RTA-IT (Xomazyme-CD5) to treat corticosteroid-resistant GVHD (39). The initial results were very promising with skin, gastrointestinal tract, and liver disease responding in 73%, 45%, and 28% of cases, respectively (39). However, more recent clinical trials have shown that Xomazyme CD5 is no more effective than alternative treatments (18). Consequently, the further development of Xomazyme-CD5 has been abandoned.

Encouraged by the initial success of the IT-based treatment of GVHD, we set up to develop alternative ITs with superior anti-T cell activity. In order to achieve this, RTA was conjugated to a panel of MoAbs that react with antigens that are expressed almost exclusively on T cells, namely the T cell differentiation-antigens CD3, CD5 and CD7, and each was assayed for its anti-T cell activity. From this preclinical study, it appears that a cocktail of SPV-T3a-RTA (CD3-IT) and WT1-RTA (CD7-IT) has the highest potential for treating patients with severe GVHD. This mixture affords:
Synergistic cytotoxicity in which the simultaneous incubation of half the effective individual dose of SPV-T3a-RTA and WT1-RTA is more effective in eliminating T cells than either IT alone (including the CD5-IT).
Broad mechanism of action in which binding of SPV-T3a to the T-cell receptor/CD3 complex results in an additive immunosuppressive effect by blocking the recognition by the donor T cells of the foreign patient antigens. This effect is independent of action of ricin A. Moreover, the binding of this particular CD3-MoAb does not stimulate T cells to produce cytokines which would otherwise augment the severity of GVHD.
Broad spectrum reactivity by which WT1-RTA is also reactive against NK cells. These lymphocytes are thought to aggravate the severity of GVHD, especially in the later phase of the disease.

It has been previously described that the use of combinations of ITs can strongly enhance the efficacy of target cell elimination. The most obvious advantage over single-IT-treatment is that fewer target cells will be multiple antigen-negative than single antigen-negative. In addition, those cells which do express substantial levels of all target-antigens might be loaded with IT to a higher extent. When the respective ITs follow a different intracellular routing, the chance of escaping therapy might be further reduced. With respect to the use of anti-T cell IT, reports addressing the combination or cocktail approach have thus far focused on in vitro applications, including the purging of bone marrow grafts. For one aspect of the present invention, we state that a mixture of murine mAb SPV-T3a (CD3, IgG2b) and WT1 (CD7, IgG1), both conjugated to dgA, forms a superior combination for the elimination or suppression of unwanted (e.g., over-reactive, misdirected, or malignant) T cells and/or NK cells. This particular combination affords important benefits which surpass the “common” synergism as observed with the more or less “random” combinations of anti-T cell IT.

Some aspects delineating the superior characteristics of this particular combination are described below.

(A) Antigen binding of a CD3 or a T cell receptor (TCR) mAb results in at least partial blocking and modulation (internalization or shedding) of the CD3/TCR-complex, thereby preventing alloactivation of the T lymphocytes.

(B) Binding of a CD3 or a TCR mAb results in at least partial Fas-mediated apoptosis of a significant fraction of activated T lymphocytes, according to a mechanism described as activation induced cell death (AICD).

These two effector mechanisms, which are independent of a conjugated toxin, are of vital importance when intervening in an acute life-threatening situation such as refractory GVHD. The temporal (blocking and modulation of CD3/TCR) as well as limited (AICD) nature of these effects only stresses the benefit of making a “real killer” of the mAb by conjugating it to a toxin. One reason we selected SPV-T3a as CD3 mAb is that SPV-T3a is an IgG2b-isotype and out of the majority of the T cells isolated and examined from the human population SPV-T3a has proven not to induce cytokine release. As a consequence, the risk of the so called “cytokine release syndrome,” which severely complicates the immunological disorder to be treated, is strongly reduced.

(C) The presence of the CD7-IT in the IT-cocktail is essential, apart from the above mentioned “common” synergism, in that it broadens the spectrum reactivity of the IT-cocktail. The CD7 antigen is also expressed on NK cells which, accordingly, form a target for this particular IT-cocktail as well. Our changed insight regarding the processes underlying GVHD is that NK cells play a distinctive role in the pathophysiology of GVHD, particularly in the efferent phase of the disease.

Clinical History of the IT-Cocktail Components:

SPV-T3a: SPV-T3a is a mouse IgG2b MoAb directed against the human T cell differentiation antigen CD3 (40).

Anti-CD3 antibody therapy is often associated with the cytokine release syndrome caused by the binding to the T cell receptor/CD3 complex (41-44). One of the important benefits of SPV-T3a is that this particular MoAb does not induce cytokine release because it is an IgG2b-isotype (45, 46).

At the Department of Hematology of the University Hospital Nijmegen, part of the bone marrow obtained from HLA-matched unrelated donors is currently treated ex vivo with a cocktail of SPV-T3a-RTA and WT1-RTA in order to eliminate immunocompetent T cells. The patients transplanted with this marrow showed normal hematopoetic reconstitution without any signs of toxicity (n=3, data not shown).

WT1: MoAb WT1 is a mouse MoAb of IgG2a isotype directed against the human T cell differentiation antigen CD7 (47, 48).

At the Department of Nephrology of the University Hospital Nijmegen, three patients who underwent a kidney-transplantation, have been treated with WT1 in order to treat an acute rejection. The administration of unconjugated WT1 appeared to be safe and did not result in either an allergic reaction or severe toxicities. No clinical efficacy could be noted.

WT1 has been conjugated to dgRTA and administered to rhesus monkeys to test its suitability for use in the therapy of leukemic meningitis (49). The major conclusion of this study was that WT1-dgRTA may be safely administered intrathecally to rhesus monkeys and could be a good candidate for the treatment of T-lymphoblastic CNS leukemia.

At the Department of Hematology of the University Hospital Nijmegen, WT1-RTA has been used since 1986 for the ex vivo purging of autologous BM of patients suffering from high-risk T cell leukemia/lymphoma in order to eliminate residual malignant cells. After purging, neither neutrophil engraftment nor immunological reconstitution was delayed (n=20) (25).

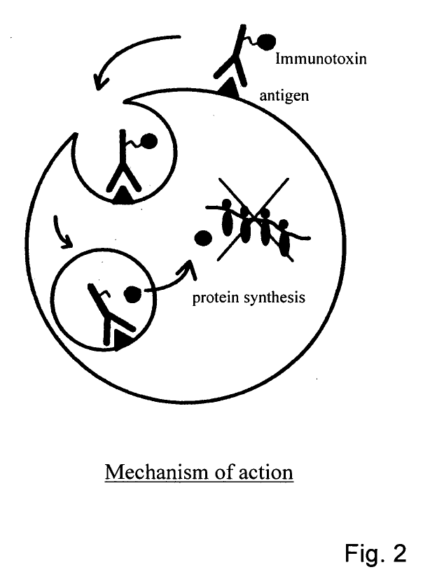

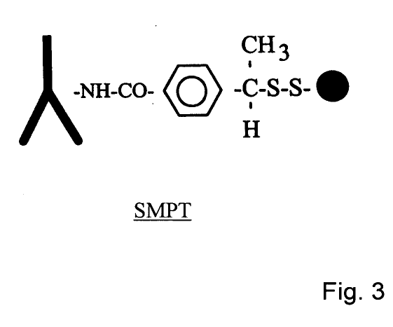

SMPT cross-linker: The MoAbs are conjugated to dgRTA using the chemical cross-linker SMPT (FIG. 3). The cross-linker contains a disulfide bond which is important for the intracellular dissociation of the MoAb and dgRTA which is necessary for toxicity. (See FIG. 2).

SMPT is a so called “second-generation cross-linker,” characterized by having a hindered disulfide bond due to the presence of the phenyl ring. This renders the SMPT-linker less susceptible to extracellular reduction by thiols present in the tissues and blood, and, therefore, results in a prolonged serum half-live of the IT. Thorpe et al. demonstrated in an in vivo mice tumor model that using SMPT instead of the first-generation cross-linker SPDP, strongly improves the anti-tumor effect of their dgRTA-based ITs (20). Amlot et al. performed a Phase I trial in which they studied the treatment of malignant lymphoma by intravenous administration of a SMPT-conjugated IT (RFB4 [IgG]-dgRTA) (50). Due to the long serum half-live of 7.8 hours, therapeutic serum concentrations could be maintained between the infusions given at 48-hour intervals.

dgRTA: The earliest RTA-based ITs consisted of a MoAb conjugated to native RTA. The oligosaccharides present on the native RTA resulted in rapid hepatic clearance and hepatotoxicity in vivo (20, 51). This problem has been addressed in the second-generation ITs which make use of either deglycosylated RTA (dgRTA) or non-glycosylated recombinant ricin A (rRTA) (52, 53).

Vitetta and colleagues have reported the administration of dgRTA-based ITs to patients with refractory B-cell non-Hodgkin's lymphoma. They tested two different constructs. In the first, the Fab′ fraction of MoAb RFB4 (anti-CD22) was conjugated to dgRTA (50). In the latter construct they used RFB4 whole molecule (54).

The ITs were administered by 4-hour intravenous infusions given at 48-hour intervals. The Phase I dose limiting toxicities included pulmonary edema, expressive aphasia, and rhabdomyolysis with acute renal failure. Other side effects included hypoalbuminemia, weight gain, fever, tachycardia, decrease in electrocardiogram voltage, myalgias, anorexia, and nausea. The maximum tolerated dose (MTD) was 75 mg/m² for the Fab′-dgRTA and 32 mg/m² for whole IgG-dgRTA. The MTD appeared to be inversely related to the serum half-life of 86 minutes and 7.8 hours, respectively. The two forms of the dgRTA-IT demonstrated no significant difference in clinical responses (partial and complete responses in 45% of the patients receiving greater than 50% of the MTD), in immunogenicity or in the toxic side effects. Because of its lower costs, the IgG-dgRTA IT was selected for further development.

The major findings to be learned from these studies are: a) The MTD of dgRTA-ITs is dependent primarily on the size of an individual dose rather than the cumulative dose. When administering RFB4 (IgG)-dgRTA at 48-hour intervals at doses of 8 mg/m² or less, only grade I or II toxicities were observed. Total doses of 32 mg/m² RFB4 (IgG)-dgRTA were consistently safe. As a consequence of the relatively long T_{1/2}, therapeutic serum concentrations (about 1.8 μg/ml) could be maintained during and between infusions. b) Side effects of the dgRTA-ITS administration were relatively modest and consisted predominantly of VLS and myalgia. No hepatotoxicity and minimal BM toxicity was observed. c) Patients with underlying pulmonary disease should not be treated because of the danger of VLS contributing to further pulmonary insufficiency.

Patients received four doses of IT-cocktail administered intravenously in 4-hour infusions at 48-hour intervals. If no clinical response was observed and if no severe toxicities (grade III or IV) occurred, the study continued with the next higher protein dose level.

Patient Population

Patients have received second-line high dose corticosteroid therapy (methylprednisolone 1000 mg/d) for at least three days without any decrease in the severity of GVHD.

Patients are EXCLUDED from participation in the study if:
1. The patient has a significant history or current evidence of intrapulmonary disease.
2. The patient has a history of allergy to mouse immunoglobulins or ricin.
3. The patient has circulating high levels of human anti-mouse antibodies (HAMA).
   Treatment
   Pharmacological Information:

The IT-cocktail has been prepared by the Department of Hematology under supervision of the Department of Clinical Pharmacy of the University Hospital Nijmegen. The IT-cocktail is stored at −80° C. at 1 mg/ml in 0.15 M NaCl, in lots of 5 and 20 mg. Before infusion, the IT-cocktail will be filtered through a 0.22 μm filter and diluted to a final volume of 100 ml in normal saline solution. The ID₅₀ against the T cell line Jurkat is taken as the standard for biological activity when evaluating the quality of different lots of IT-cocktail.

Immunotoxin Administration:

Immunotoxins are administered via a central venous catheter. Prior to therapy, patients are given an intravenous test done with 200 μg IT-cocktail. Therapy is only started in the absence of anaphylactoid reactions. The IT-cocktail is administered in four doses at 48-hour intervals. The rationale of this is to give all of the IT-cocktail before any host antibody response is expected to arise (usually not before 10 to 14 days after administration of xenogenic Ig).

The patient is initially treated with two subsequent doses of 2 mg/m². At this dose level no side effects are observed. In the absence of grade III or IV toxicities, the dose will be enhanced to 4 mg/m² if necessary.

Guidelines for Dose Modification:

Toxicities related to the immunotoxin administration are graded as grade I (mild), II (moderate), III (severe) or IV (life threatening) based on World Health Organization (WHO) guidelines. Special attention must be paid to the vascular leak syndrome (VLS). The physical signs of VLS are weight gain, peripheral edema, decrease in blood pressure, hypoalbuminemia, and small pleural effusions.

Consecutive doses given to the same patient: Infusion of the second, third and fourth dose at any dose level is dependent upon the toxicity achieved after the previous infusion:
Grade I toxicity: no change in the scheduled dosage.
Grade II toxicity: 24 hours-delay of the dosage, with the next dose given if toxicity improves.
Grade III toxicity: the next dosage of immunotoxin will be withheld and only given if toxic parameters have improved (halving of the dosage can be considered).
Grade IV toxicity: no further dosage.

Dose escalation: Progression from one dose level to the next should only occur after:
The patient(s) of the group treated with the previous dose level have received four doses of IT-cocktail and have been observed for at least 48 hours after the last dose; and
Dose limiting toxicity has not been reached.

Dose limiting toxicity: Dose limiting toxicity is defined as the occurrence of adverse reactions of grade III or IV in an individual patient. If two patients experience a Grade III toxicity or if Grade IV toxicity occurs in a single patient, three additional patients will be entered at this dose level. If none of these additional patients demonstrate toxicity of grade III or IV, administration will again be continued to the next higher dose level. If Grade IV toxicity occurs in two patients at a given dose, the next patients will be treated with the previous dose level which will be considered the Maximum Tolerable Dose (MTD).

Concomitant Medication and Treatment:

Immunosuppressive agents used for prophylaxis and initial treatment may maintain unchanged throughout immunotoxin therapy.

Pretreatment Studies:

Before entry into the study, the patient undergoes a general examination consisting of medical history, physical examination with special emphasis on acute GVHD, measurement of oxygen saturation, electrocardiogram (ECG), and chest x-ray. The laboratory measurements will include Na+, K+, Cl—, HCO₃—, urea, creatine, bilirubin, glucose, AP, ASAT (GOT), ALAT (GPT), gGT, LDH, total protein plus electrophoresis, leukocytes plus differentiation, red cells, hemoglobin, hematocrit, and thrombocytes.

The patient is assayed for human anti-mouse and anti-ricin responses (HAMA/HARA), and serum levels of IL-2, TNF-α and IFN-γ. Furthermore a quantitative alloreactive T-helper/T-cytotoxic precursor assay will be performed.

Follow-Up Studies:

Daily complete physical examinations are performed during IT-cocktail administration until two days after the last dose and weekly thereafter. Blood is analyzed daily for Na+, K+, Cl—, HCO₃—, urea, creatine, glucose, albumin, leukocytes plus differentiation, red cells, hemoglobin, hematocrit, and thrombocytes. Blood is analyzed every two days for bilirubin, AP, ASAT (GOT), ALAT (GPT), gGT, LDH, total protein plus electrophoresis.

Vital signs (blood pressure, pulse, respiration frequentation, and temperature) are checked every 15 minutes during the first hour post infusion, every 30 minutes during the second up to fourth hour post infusion, and from then on every hour up to eight hours post infusion. In addition, vital signs are assessed daily during IT-cocktail administration until two days after the last dose, and then weekly thereafter.

Pharmocokinetics and clearance of Its: Blood samples are collected at 0, 1, 3, 4, 8, 12, 24, and 48 hours after each infusion. The serum concentrations of SPV-T3a-dgRTA and WT1-dgRTA are quantitatively determined in a sensitive and mAb-isotype specific immuno-radiometric assay (IRMA). From these results the individual serum half-lives of the two are calculated using non-linear, least squares regression analysis.

Measurement of humoral responses: In order to examine HAMA and HARA responses, serum samples are obtained one day pre-injection and weekly after the first infusion until the patient comes off the study. The concentration of HAMA and HARA is determined in a sensitive radiometric assay.

Immunological monitoring: Blood is sampled every other day during IT-cocktail administration until two days after the last dose and then weekly thereafter for immunological monitoring. PBLs are isolated and evaluated for composition by flow cytometry using antibodies reacting specifically with T cells subsets, B cells, monocytes/macrophages, and NK cells. Serum is collected to determine levels of IL-2, TNF-α and IFN-γ by commercial enzyme-linked immunosorbent assay (ELISA) kits.

The proliferative and cytotoxic activity of alloreactive PBLs is tested two days following the last dose using standard T-helper and T-cytotoxic-precursor assays, respectively.

Staging and Grading of GVHD and clinical responses: GVHD is scored daily during IT-cocktail administration until two days after the last dose, and weekly thereafter until the patient comes off the study. Each organ system is evaluated grade I through IV GVHD according to the criteria of Glucksberg et al.: skin by amount of surface involved with rash, gastrointestinal tract by the volume of diarrhea, and liver by serum bilirubin levels. Patients are also given an overall grade of GVHD based on severity of organ involvement.

Responses to therapy are defined as follows:
complete response (CR): the disappearance of symptoms in all organ systems;
partial response (PR): improvement of >1 organ, with no worsening in other organs.
mixed response (MR): improvement of >1 organ, with worsening in >1 other organ.
stable disease: no significant change in any organ system.
progressive disease (PD): progression in >1 organ system without improvement in any organs.

The duration of response is defined as the period from the date the response was first recorded to the date on which subsequent progressive disease is first noted.

Results

Animal Toxicity Studies and Preclinical Studies

Animal toxicity studies and preclinical studies are summarized herein.

Inhibition of Alloactivation by Unconjugated SFV-T3a

Method:

Alloactivation was analyzed in a mixed lymphocyte culture (MLC). MLC were performed with “responder” peripheral blood lymphocytes (PBL) mixed in a one to one ratio with irradiated “stimulator” PBL. Cultures were performed in triplicate (5×10⁴ cells/well) in U-bottomed microtiter plates in 150 μl culture medium at 37° C. and 5% CO₂. Prior to, or at different days following initiation of the MLC, SPV-T3a (10⁻⁸ M) or an irrelevant isotype-matched control antibody were added to the culture medium. Following 72 hours of culture, plates were labeled with [³H]thymidine (0.4 μCi/well) for 4 hours. Subsequently, the proliferation of responder cells was determined by collecting the DNA using a cell harvester and counting the incorporated radioactivity. Proliferation was expressed as a percentage of the untreated control.

Results:

Alloactivation was completely blocked when SPV-T3a was added directly following the initiation of the MLC. When addition of SPV-T3a was postponed to one or more days following initiation, this effect gradually ceased to exist. Following four days, addition of SPV-T3a no longer had an effect on proliferation. The irrelevant isotype-matched control antibody did not influence alloactivation at all time points. Translated to the in vivo situation these results demonstrate that unconjugated SPV-T3a is capable of delivering a direct and important immunosuppressive effect by preventing ongoing allostimulation of T lymphocytes. For the suppression or elimination of already stimulated T lymphocytes, SPV-T3a is dependent on another effector mechanism, termed activation induced cell death (AICD), or needs to be conjugated to a toxin.

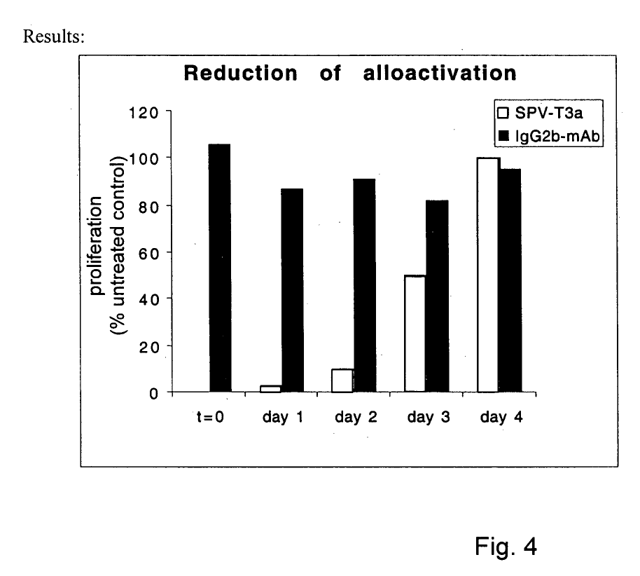

The results are summarized in graphical form in FIG. 4.

Activation Induced Cell Death by Unconjugated SPV-T3a

Method:

Reduction of TCR-mediated cytotoxicity following IT-treatment was assayed in vitro using a cytotoxic T cell clone (CTL-clone) recognizing EBV-peptide EBNA3C presented in HLA-B44. CLT activity was assayed by lysis of a loaded EBV-transformed lymphoblastoide cell line (EBV-LCL) originating from the same individual. The CTL-clone was treated for 24 hours with SPV-T3a, washed and assessed either directly or following four days of additional incubation in culture medium. The extended four day incubation period was incorporated since during this time the CTL-clone restored its normal expression of the TCR/CDS complex (which is blocked and/or modulated directly following incubation with SPV-T3a).

Results:

Directly following treatment (day 1), incubation with native mAb SPV-T3a (108 M) resulted in a modest reduction of CTL-cytotoxicity. Flow cytometric analysis revealed that this effect was predominantly caused by the blocking and modulation of the TCR/CD3 complex due to binding of SPV-T3a. Following four days of extended incubation, the CTL-clone regained its normal TCR/CD3-expression, but CTL-cytotoxicity was further reduced to 18% of the untreated control (day 5). This time, flow cytometric analysis revealed that the majority of the CTL-cells had died due to apoptosis, according to the mechanism described as “activation induced cell death” (AICD). Translated to the in vivo situation this means that unconjugated SPV-T3a is capable of delivering an important immunosuppressive effect by eliminating a significant fraction of activated T lymphocytes. The efficacy of SPV-T3a will be further enhanced when conjugated to a toxin like ricin A.

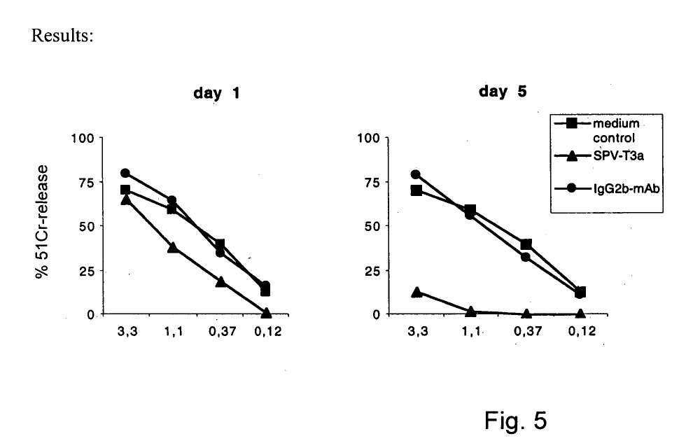

The results are summarized in graphical form in FIG. 5.

Common IT-Cocktail Synergism of SPV-T3a-dgA & WT1-dgA

Method:

PHA-stimulated PBL were treated with 10⁸ M IT for 24 hours at 37° C., washed, and cultured for another four days at 37° C. in IT-free culture medium (to enable the IT to display their full toxicity). After this lag period, cells were incubated with 2 μg/ml propidium iodine (PI) (Molecular Probes, Junction City, Oreg.) and 2 μg/ml calcein AM (Calc) (Molecular Probes) for 1 hour at RT. Samples were then analyzed on a Coulter Epics Elite (Coulter) flow cytometer equipped with a 40 mW Argon ion laser running at 15 mW. A longpass-filter of 610 nm was used for measurement of PI-fluorescence, a bandpass-filter of 525/30 nm for Calc-fluorescence. Overlap of the emission spectra of PI and Calc could be adjusted by electronic compensation using single-labeled samples. Samples were analyzed in triplicate using a minimum of 10,000 cells. Viable cells were identified as being PI-negative and Calc-positive. Prior to FCM analysis, a fixed amount of inert beads (DNA-check, Coulter) was added (10⁵ beads/ml) to enable the calculation of the number of surviving cells. The reduction of PBL was related to the viable fraction of the untreated control.

Results:

| Treatment | Factor of PBL reduction |
|---|---|
| SPV-T3a-dgA | 100 |
| WT1-dgA | 87 |
| SPV-T3a-dgA and WT1-dgA | 1770 |
| (half a dose each) | |

Due to the “common IT-cocktail synergism” IT SPV-T3a-dgA and WT1-dgA appeared to be far more effective in combination (half a dose each) than either IT alone.

Reduction of Natural Killer Activity by WT1-dgRTA

Method:

Blood mononuclear cells were isolated from peripheral blood by Ficoll centrifugation and incubated with 108 M mAb or IT in a concentration of 1×10⁶/ml for 24 hours. Subsequently, cells were washed and analyzed for NK-activity after 4 additional days of incubation without IT (this lag period is essential for IT to display their full efficacy). During the experiment, 50 units/ml recombinant IL2 was added to the culture medium to increase NK-activity. For analysis of NK-activity, cells were serial diluted and incubated with a fixed number of ⁵¹Cr-labeled K562 blasts (10⁴/100 μl) to yield an effector to target ratio of 10:1, 3.3:1, 1.1:1, and 0.37:1. After 3.5 hours of incubation at 37° C., the cell mixtures were centrifuged and radioactivity was measured. NK-activity was expressed as a percentage maximum ⁵¹Cr-release as determined with saponin treated ⁵¹Cr-labeled K562 blasts. Both were corrected for spontaneous ⁵¹Cr-release as determined with ⁵¹Cr-labeled K562 blasts incubated with culture medium only.

Results:

Incubation with saturating amounts of mAb SPV-T3a (10 μg/ml) had no effect on the NK-activity, nor had treatment with SPV-T3a-dgA (10⁻⁸ M). Four days following incubation with WTI-dgA, in contrast, the NK-activity distinctively reduced to 8% of the untreated control. Neither unconjugated WT1, nor the isotype-matched control IT influenced the NK-activity. Translated to the in vivo situation, this means that incorporation of WT1-dgA in the IT-cocktail not only results in the common IT-cocktail synergism, but also broadens the spectrum reactivity. This is of vital importance since, though initiated by CTL, GVHD is thought to be aggravated by less specific cytokine-stimulated bystander cells like monocytes and NKILAK cells.

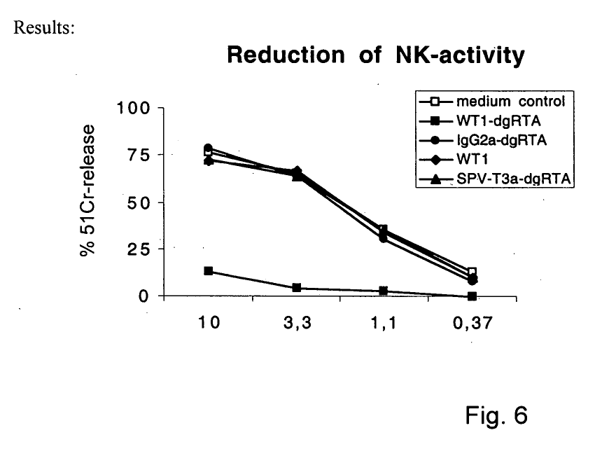

The results are summarized in graphical form in FIG. 6.

Animal Toxicity Studies:

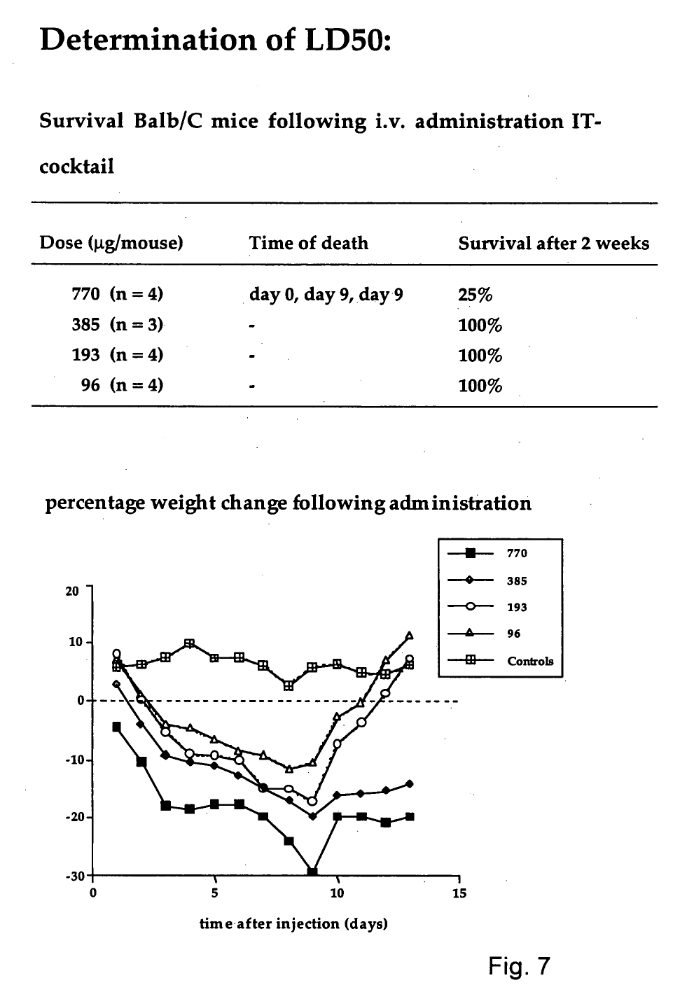

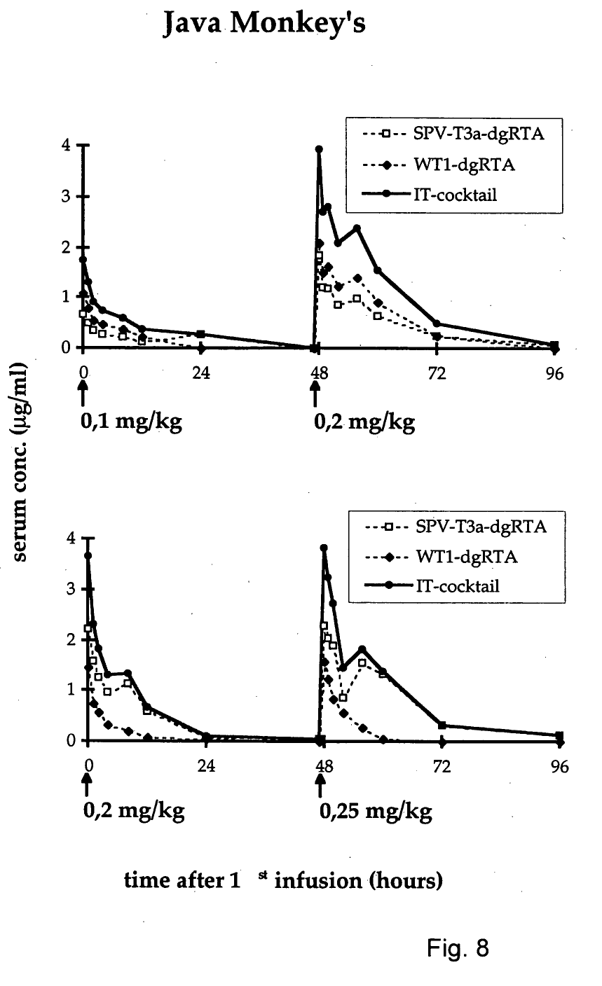

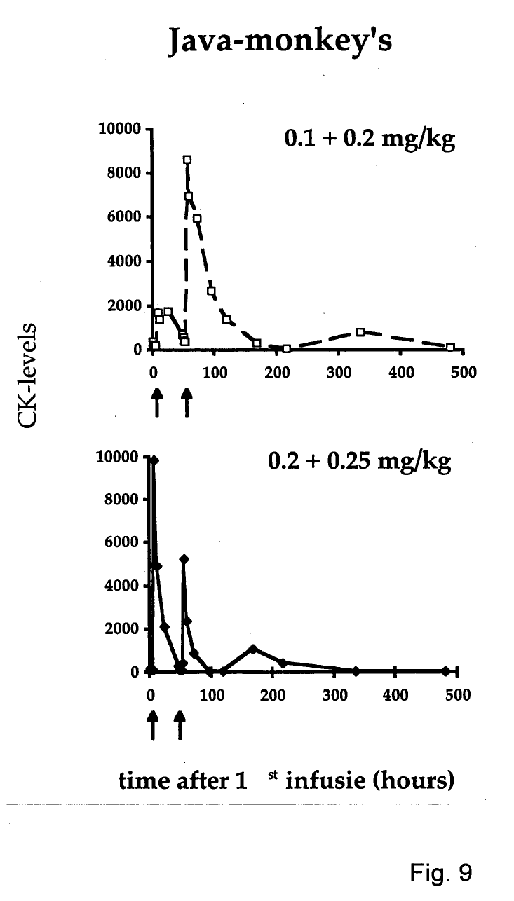

A. LD5O determination with Balb/C mice (See, FIG. 7):
   IT-cocktail: 25-45 mg/kg
   RFB4-dgA (based on literature): 14 mg/kg
B. Administration to Java-monkeys (See, FIGS. 8 and 9):
   Rise of CK-levels following infusion
   No further acute toxicities
      Clinical Study.

The results of the clinical study are reported herein.

Clinical Pilot-Study:

Ongoing one center, non-randomized, open labeled, dose escalating study (aim of treating 5-7 patients)

FOur doses intravenously at 48-hour intervals:

| | Dose of IT-cocktail | | | | |
|---|---|---|---|---|---|
| | (mg/m²) | | | | |
| #PATIENTS | D1 | D3 | D5 | D7 | Total |
| 1 | 2 | 2 | 4 | 4 | 12 |
| 2 | 4 | 4 | 4 | 4 | 16 |
| 2 | 8 | 8 | 8 | 8 | 32 |
| 2 | 10 | 10 | 10 | 10 | 40 |

Evaluation: pharmacokinetics, toxicities, human-anti-mouse antibodies and human-anti-ricin antibodies (HAMA and HARA), biological and clinical responses

First Patient Characteristics:
Male 60, Multiple Myeloma
Sibling transplantation
GVHD of skin, gut and liver (overall grade IV)
Complication: multi-organ failure
IT-cocktail: 2 doses 2 mg/m², 1 of 4 mg/m²
Died seven days following the first infusion

First Patient, Toxicities:
Mild capillary leakage, no weight gain
No increase of CK-levels
No further acute toxicities
No HAMAs/HARAs

First Patient Clinical Response:
Skin: improvement starting at day 5
Liver: stable (poor condition)
Gastrointestinal tract: not interpretable (morphine)

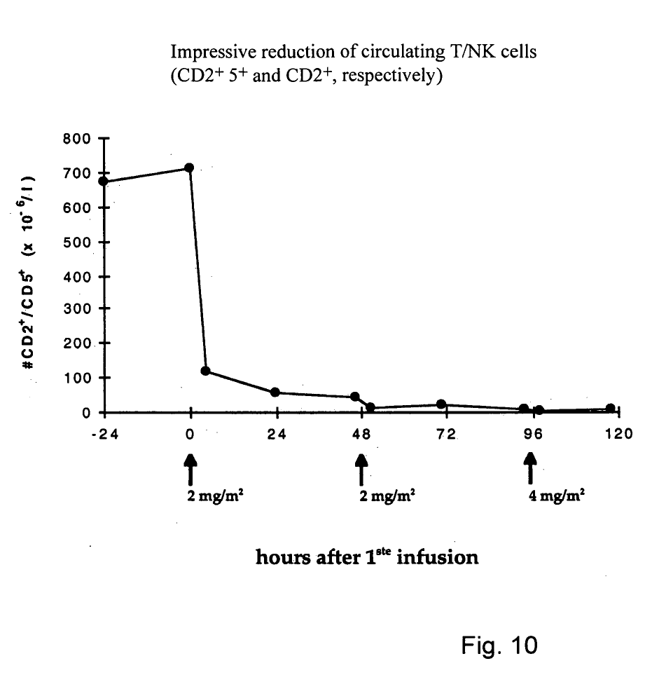

First Patient, Biological Response: (See, FIG. 10)
Impressive reduction of circulating T/NK cells (CD2+5+ and CD2+, respectively)
During first 4 hour infusion: decrease to 17%
Gradually declines further to 1% at day 7
Dual mechanism: mAb-based (fast) & dgA-based (lasting)

Second Patient, Characteristics:
Male 34, CML
Matched unrelated donor
Grade 4 GVHD of the skin
IT-cocktail: 2 doses 2 mg/m², 2 of 4 mg/m²

Second Patient, Toxicities:
No acute toxicities could be observed
No HAMAs/HARAs

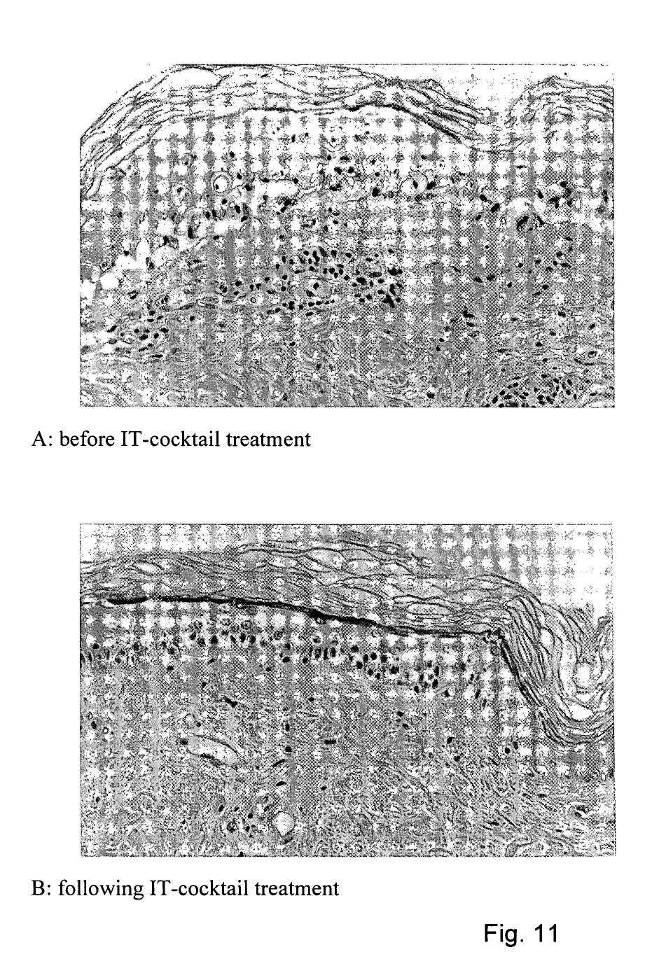

Second Patient, Responses (See, FIG. 11):
Dramatic (complete) response starting at day 5
Lasting for—1.5 month
Relapse of GVHD I-II
Responding to low dose corticosteroids
Died 8 months following treatment due to an infection

Third Patient, Characteristics:
Male 47, MDS
HLA-identical donor
GVHD grade ¾ of the gut
IT-cocktail: 4 doses of 4 mg/m²

Third Patient Toxicities:
Rise in body temperature during infusions
No further acute toxicities
No HAMAs/HARAs

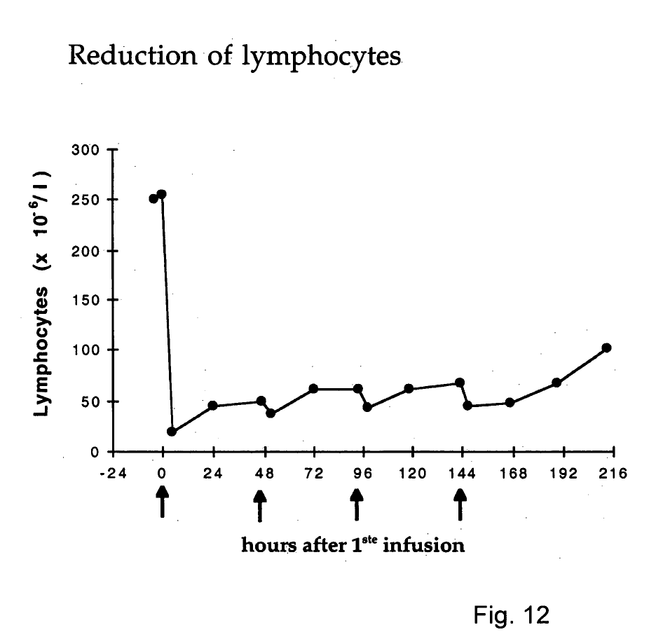

Third Patient Responses:
Reduction of lymphocytes (see, FIG. 12)
Decrease of stool volume
Endoscopy: strong improvement of gut-tissue

Conclusions Pilot-Study:
IT-cocktail is well tolerated, no acute severe toxicities extensive biological and clinical responses in the absence of acute severe toxicities
IT-cocktail forms effective tool for in vivo suppression or elimination of misdirected, over-reactive or malignant T cells and/or NK cells
   Materials and Methods.
   Making Monoclonal Antibodies

The monoclonal antibodies disclosed herein are prepared using hybridoma technology well known in the art. Selection steps to select immunoglobulins having the properties disclosed hereinabove are also well known in the art and may include affinity chromatography and the like.

Preparation of F(ab′)₂ Fragments

F(ab′)₂ fragments of antibodies were prepared using a “F(ab′)₂ preparation kit” (Pierce, Rockford, Ill.), according to the manufacturer's protocol. Briefly, antibodies were incubated with immobilized pepsin at pH 4.2 (20 mM sodium acetate buffer) for four and sixteen hours, respectively. Undigested IgG molecules, and Fc-fragments were removed by affinity chromatography with protein A-sepharose. Remaining fragments smaller than 30 kD were removed by means of a centriprep-30 concentrator (Amicon, Beverly, Mass.). The purity of F(ab′)₂ fragments was determined by SDS-PAGE, which revealed less then 1% contamination with either intact IgG or Fc-fragrnents (data not shown).

IT Preparation.

Antibodies were conjugated on a 1 to 1 ratio (m/m) to deglycosylated ricin A (dgA, Inland Laboratories, Austin, Tex.) using the SMPT-cross-linker (Pierce, Rockford, Ill.), according to the method as described by Ghetie et al. (Ghetie, V. et al., The GLP large scale preparation of immunotoxins containing deglycosylated ricin A chain and a hindered disulfide bond, J. Immunol. Methods 1991, 142:223-30).

Antibodies were conjugated to ricin A (kindly provided by Dr. F. K. Jansen; Centre de Recherches Clin. Midy, Montpellier, France) using N-succinimydyl 3-(2-pyridyldithio) propionate (SPDP; Pharmacia) or SMPT, as described. (The conjugation ratios of ricin A to mAb were estimated by measurement of absorbance at 280 nm and RIA, and were determined to be in the order of 0.8 to 1.2.) Preservation of antibody-binding activity following conjugation was assessed by FCM.

TABLE I
Dose levels:

| | Dosage of IT- | | | | |
|---|---|---|---|---|---|
| | cocktail | | | | |
| | (mg/m²) | | | | |
| Patients | d1 | d3 | d5 | d7 | Total |
| 1 | 2 | 2 | 4 | 4 | 12 |
| 2 | 4 | 4 | 4 | 4 | 16 |
| 2 | 8 | 8 | 8 | 8 | 32 |
| 2 | 10 | 10 | 10 | 10 | 40 |

TABLE 2
Flow-chart of IT-cocktail study (summary of study requirements):

| | PROTOCOL FLOW SHEET | | | | | | | | | | | | | | |
|---|---|---|---|---|---|---|---|---|---|---|---|---|---|---|---|
| | (days following initiation of the study) | | | | | | | | | | | | | | |
| | | 1st | | 2nd | | 3rd | | 4th | | | | | | | |
| | | inj. | | inj. | | inj. | | inj. | | | | | | | |
| | | ↓ | | ↓ | | ↓ | | ↓ | | | | | | | |
| | Pre | d1 | d2 | d3 | d4 | d5 | d6 | d7 | d8 | d9 | d10 | d22 | d29 | d36 | d43 |
| Informed consent | 0 | | | | | | | | | | | | | | |
| History and | 0 | 0 | 0 | 0 | 0 | 0 | 0 | 0 | 0 | 0 | 0 | 0 | 0 | 0 | 0 |
| Phys. exam. | | | | | | | | | | | | | | | |
| Chest x-ray | 0 | | | | | | | | | | | | | | |
| ECG | 0 | | | | | | | | | | | | | | |
| Vital signs | | 0¹ | 0 | 0¹ | 0 | 0¹ | 0 | 0¹ | 0 | 0 | 0 | 0 | 0 | 0 | 0 |
| Biochemistry² | 0 | 0 | 0 | 0 | 0 | 0 | 0 | 0 | 0 | 0 | 0 | 0 | 0 | 0 | 0 |
| Hematology³ | 0 | 0 | 0 | 0 | 0 | 0 | 0 | 0 | 0 | 0 | 0 | 0 | 0 | 0 | 0⁴ |
| Pharmokinetics | | 0⁵ | | 0⁵ | | 0⁵ | | 0⁵ | | | | | | | |
| HAMA/HARA | 0 | | | | | | | | 0 | | 0 | 0 | 0 | 0 | 0 |
| Flow cytometry 6 | 0 | | 0 | | 0 | | 0 | | 0 | 0 | 0 | 0 | 0 | 0 | 0 |
| Cytokine levels⁷ | 0 | | 0 | | 0 | | 0 | | 0 | 0 | 0 | 0 | 0 | 0 | 0 |
| Allo-precursors | 0 | | | | | | | | 0 | | | | | | |
| GVHD staging | 0 | 0 | 0 | 0 | 0 | 0 | 0 | 0 | 0 | 0 | 0 | 0 | 0 | 0 | 0⁸ |
| ¹Vital signs are checked every 15 minutes during the first hour post-injection, every 30 minutes during the second up to the fourth hour, and from then on every hour up to eight hours post-injection. | | | | | | | | | | | | | | | |
| ²The pre-study biochemistry panel includes Na+, K+, Cl−, HCO₃−, urea, creatine, bilirubin, glucose, AP, ASAT (GOT), ALAT (GPT), γGT, LDH, and total protein plus electrophoresis. During the follow-up study, blood will be analyzed daily for Na+, K+, Cl−, HCO₃−, | | | | | | | | | | | | | | | |
| # urea, creatine, glucose, and albumin. Besides, every two days is added bilirubin, AP, ASAT (GOT), ALAT (GPT), γGT, LDH, and total protein plus electrophoresis. | | | | | | | | | | | | | | | |

³The hematology panel includes leukocytes plus differentiation, red cells, hemoglobin, hematocrit, thrombocytes.
⁴To be continued weekly until WBC numbers have returned to normal.
⁵Venous blood samples are obtained pre-injection and 1, 3, 4, 8, 12, 24, and 48 hours after each injection. Besides, a sample is taken 72 hours following the last injection.
⁶The flow cytometry panel includes the markers CD2, CD3, CD4, CD5, CD7, CD8, CD14, CD19 and CD56.
⁷Serum is assayed for levels of IL-2, TNF-α and IFN-γ.
⁸To be continued monthly when responses are observed.

TABLE 3
staging acute graft-versus-host disease (GVHD)
(Glucksberg et al, Transplantation 1974, 18: 295-304)

Skin.
grade 1: maculo-papular eruption involving less than 25% of the
body surface
grade 2: maculo-papular eruption involving less than 25% to 50%
of the body surface
grade 3: generalized erythroma
Liver
grade 1: bilirubin 34-50 μmol/l
grade 2: bilirubin 51-100 μmol/l
grade 3: bilirubin 101-254 μmol/l
grade 4: >255 μmol/l
Gut
grade 1: 500 to 1000 ml of stool/day
grade 2: 1001 to 1500 ml of stool/day
grade 3: 1501 to 2000 ml of stool/day
grade 4: >2000 ml of stool/day
Overall severity of GVHD
grade 1: skin: 1/2; gut: 0; liver 1
grade 2: skin: 1/2/3/; gut 1/2 and/or liver 1/2 sometimes associated
with fever
grade 3 skin: 2/3/4; gut 2/3/4 and/or liver 2/4 often associated with fever.
grade 4: similar to grade 3 but extreme constitutional symptoms

### REFERENCES

1. Storb R., Thomas E. D. Human marrow transplantation. Transplantation 1979,28:1.
2. O'Reilly R. J. Allogeneic bone marrow transplantation: current status and future directions. Blood 1983, 62:941.
3. Goldman J. M., Apperley J. F., Jones L., et al. Bone marrow transplantation for patients with chronic myeloid leukemia. N. Engl. J. Med. 1986, 314:202.
4. Champlin R. Bone marrow transplantation for acute leukemia: a preliminary report from the International Bone Marrow Transplant Registry. Transplant Proc. 1987, 19:2626.
5. Ringden O., Sundberg B., Lonnqvist B., et al. Allogeneic bone marrow transplantation for leukemia: factors of importance for long-term survival and relapse. Bone Marrow Transplant 1988, 3:281.
6. Snyder D. S., Findley D. O., Forman S. J., et al. Fractionated total body irradiation and high dose cyclophoshamide: a preparative regimen for bone marrow transplantation for patients with hematologic malignancies in first complete remission. Blut. 1988, 57:7.
7. Martin P. J., Hansen J. A., Buckner C. D., et al. Effects of in vitro depletion of T cells in HLA-identical allogeneic marrow grafts. Blood 1985, 66:664.
8. Patterson J., Prentice H. G., Brenner M. K., et al. Graft rejection following HLA matched T-lymphocyte depleted bone marrow transplantation. Br. J. Haematol. 1986; 63:221.
9. Kernan N. A., Flomenberg N., Dupont B., et al. Graft rejection in recipients of T cell-depleted HLA-nonidentical marrow transplants for leukemia. Transplantation 1987, 43:842.
10. Vallera D. A., Blazar B. R. T cell depletion for graft-versus-host-disease prophylaxis. A perspective on engraftment in mice and humans. Transplantation 1989, 47:751.
11. Horowitz M., Gale R. P., Sondel et al. Graft-versus-leukemia reactions after bone marrow transplantation. Blood 1990, 75:555.
12. Sullivan K. M., Weiden P. L., Storb R., et al. Influence of acute and chronic graft-versus-host disease on relapse and survival after bone marrow transplantation from HLA-identical siblings as treatment of acute and chronic leukemia. Blood 1989, 73:1720.
13. Truitt R. L., Lefevre A. V., Shih C. C. Graft-vs-leukemia reactions: Experimental models and clinical trials. In: Gale R. P., Champlin R. (eds). Progress in bone marrow transplantation, New York, Liss. 1987:219.
14. Glucksberg H., Storb R., Fefer A., et al. Clinical manifestations of graft-versus-host disease in human recipients of marrow from HLA-matched sibling donors. Transplantation 1974, 18:295.
15. Grebe S. C., Streilein J. W. Graft-versus-host disease. Adv. Immunol. 1976, 22:119.
16. Ferrara J. L. M., Deeg H. J. Graft-versus-host disease. N. Engl. J. Med. 1991, 304:667.
17. De Witte T., Hoogenhout J., De Pauw B., et al. Depletion of donor lymphocytes by counterflow centrifugation successfully prevents acute graft-versus-host disease in matched allogeneic marrow transplantation. Blood 1986, 67:1302.
18. Hings I. M., Severson R., Filipovich A. H., et al. Treatment of moderate and severe acute GVHD after allogeneic bone marrow transplantation. Transplantation 1994, 58:437.
19. Hervé P., Wijdenes J., Bergerat J. P., et al. Treatment of corticosteroid-resistant acute graft-versus-host disease by in vivo administration of anti-interleukin-2 receptor monoclonal antibody (B-B 10). Blood 1990, 75:1426.
20. Thorpe P. E., Wallace P. M., Knowles P. P., et al. Improved anti-tumor effects of immunotoxins prepared with deglycosylated ricin A chain and hindered disulfide linkages. Cancer Res. 1988, 48:6396.
21. Preijers F. W. M. B., Tax W. J. M., Wessels J. M. C., et al. Different susceptibilities of normal T cells and T cell lines to immunotoxins. Scand. J. Immunol. 1988a, 27:533.
22. Preijers F. W. M. B., Tax W. J. M., De Witte T. J. M., et al. Relationship between internalization and cytotoxicity of ricin A-chain immunotoxins. Br. J. Haematol. 1988b, 70:289.
23. Preijers F. W. M. B., De Witte T., Rijke-Schilder G. P. M., et al. Human T lymphocytes differentiation antigens as target for immunotoxin or complement-mediated cytotoxicity. Scand. J. Immunol. 1988c, 28:185.
24. Preijers F. W. M. B., De Witte T., Wessels J. M. C., et al. Cytotoxic potential of anti-CD7 immunotoxin (WT1-ricin A) to purge ex vivo malignant T cells in bone marrow. Br. J. Haematol. 1989a, 71:195.
25. Preijers F. W. M. B., De Witte T., Wessels J. M. C., et al. Autologous transplantation of bone marrow purged in vitro with an anti-CD7-(WT1)-ricin A immunotoxin in T cell lymphoblastic leukemia and lymphoma. Blood 1989b, 74:152.
26. Preijers, F. W. M. B. Rationale for the clinical use of immunotoxins: monoclonal antibodies conjugated to ribosome-inactivating proteins. Leukemia Lymphoma 1993, 9:293.
27. Van Horssen P. J., Van Oosterhout Y. V. J. M., De Witte T., et al. Cytotoxic potency of CD22-ricin A depends on intracellular routing rather than on the number of internalized molecules. Scand. J. Immunol. 1995, 41:563.
28. Van Oosterhout Y. V. J. M., Preijers F. W. M. B., Wessels J. M. C., et al. Cytotoxicity of CD3-ricin A chain immunotoxins in relation to cellular uptake and degradation kinetics. Cancer Res. 1992, 52:5921.
29. Van Oosterhout Y. V. J. M., Van De Herik-Oudijk I. E., Wessels H. M. C., et al. Effect of isotype on internalization and cytotoxicity of CD19 ricin A immunotoxins. Cancer Res. 1994a, 54:3527.
30. Van Oosterhout Y. V. J. M., Preijers F. W. M. B., Meijerink J. P. P., et al. A Quantitative flow cytometric method for the determination of immunotoxin-induced cell kill in marrow grafts. Advances in bone marrow purging and processing: Fourth international symposium 1994b, pages 89-95, Wiley-Liss, Inc.
31. Van Oosterhout Y. V. J. M., Van Emst L., De Witte T., et al. Suitability of a cocktail of CD3- and CD7-ricin A immunotoxins for in vivo treatment of GVHD. Abstract for the Fourth International Symposium on Immunotoxins: Jun. 8-10, 1995, Myrtle Beach, S.C.
32. Ghetie V., Vitetta E. Immunotoxins in the therapy of cancer from bench to clinic. Pharmac. Ther. 1994, 63:209.
33. Weiner L. M., O'Dwyer J., Kitson J., et al. Phase I evaluation of an anti-breast carcinoma monoclonal antibody 260F9-recombinant ricin A conjugate. Cancer Res. 1989,49:4062.
34. Pai L. H., Bookman M. A., Ozols R. F., et al. Clinical evaluation of intraperitoneal Pseudomonas exotoxin immunoconjugate of OVB3-PE in patients with ovarian cancer. J. Clin. Oncol. 1991, 9:2095.
35. Oratz R., Speyer J. L., Wemtz J. C., et al. Antimelanoma monoclonal antibody-ricin A chain immunoconjugate (XMMME-001-RTA) plus cyclophosphamide in the treatment of metastatic malignant melanoma: results of a Phase II trial. J. Biol. Resp. Med. 1990, 9:345.
36. LeMaistre C. F., Rosen S., Frankel A., et al. Phase I trial of H65-RTA immunoconjugate in patients with cutaneous T cell lymphoma. Blood 1991, 78:1173.
37. LeMaistre C. F., Deisseroth A., Fogel B., et al. Phase I trial of an interleukin-2 (IL-2) fusion toxin (DAB₄₈₆IL-2) in hematologic malignancies expressing the IL-2 receptor. Blood 1992, 79:2547.
38. Vitetta E., Thorpe P. E., Uhr J. W. Immunotoxins: magic bullets or misguided missiles? Immunol. Today 1993, 14:252.
39. Beyers V. S., Henslee P. J., Keman N. A., et al. Use of an anti-pan T-lymphocyte ricin A chain immunotoxin in steroid-resistant acute graft-verus-host disease. Blood 1990, 75:1426.
40. Spits H., Keizer G., Borst J., et al. Characterization of monoclonal antibodies against cell surface molecules associated with cytotoxic activity of natural and activated killer cells and cloned CTL lines. Hybridoma 1983, 2:423.
41. Thistlethwaite J. R., Stuart J. K., Mayes J. T., et al. Complications and monitoring of OKT3 therapy. Am. J. Kidney Dis. 1988, 11:112.
42. Ellenhom J. D. I., Woodle E. S., Ghobrial I., et al. Activation of human T cells in vivo following treatment of transplant reicpients with OKT3. Transplantation 1990, 50:608.
43. Abramowicz D., Schandene L., Goldman M., et al. Release of tumor necrosis factor, interleukin-2, and gamma-interferon in serum after injection of OKT3 monoclonal antibody in kidney transplant recipients. Transplantation 1989, 47:606.
44. Chatenoud L., Ferran C., Legendre C., et al. In vivo cell activation following OKT3 administration. Transplantation 1990, 49:697.
45. Woodle E. S., Thistlethwaite J. R., Jolliffe L. K., et al. Anti-CD3 monoclonal antibody therapy. Transplantation 1991, 52:361.
46. Frenken L. A. M., Koene R. A. P., Tax W. J. M. The role of antibody isotype in IFN-g and IL-2 production during anti-CD3-induced T cell proliferation. Transplantation 1991, 51:881.
47. Tax W. J. M., Willems H. W., Kibbelaar M. D. A., et al. Monoclonal antibodies against human thymocytes and T lymphocytes. Protides of the biologicalfluids, 29th Colloquium 1981, edited by Peeters H., Pergamon Press, Oxford and New York, 1982.
48. Tax W. J. M., Tidman N., Janossy G., Trejdosiewicz L., Willems R., Leeuwenberg J., De Witte T. J. M., Capel P. J. A., Koene R. A. P: Monoclonal antibody (WT1) directed against a T cell surface glycoprotein: characteristics and immunosuppressive activity. Clin. Exp. Immunol. 55:427, 1984.
49. Hertler A. A., Schlossman D. M., Borowitz M. J., et al. An immunotoxin for the treatment of T-acute lymphoblastic leukemic meningitis: studies in rhesus monkeys. Cancer Immunol. Immunother. 1989, 28:59.
50. Amlot P. L., Stone M. J., Cunningham D., et al. A phase I study of an anti-CD22-deglycosylated ricin A chain immunotoxin in the treatment of B-cell lymphomas resistant to conventional therapy. Blood 1993, 82:2624-2633.
51. Blakey D. C., Watson G. J., Knowles P. P., et al. Effect of chemical deglysocylation of ricin A chain on the in vivo fate and cytotoxic activity of an immunotoxin composed of ricin A chain and anti-Thy 1.1 antibody. Cancer Res. 1987, 47:947.
52. Ghetie V., Ghetie M., Uhr J. W., et al. Large scale preparation of immunotoxins constructed with the Fab′ fragment of IgG1 murine monoclonal antibodies and chemically deglycosylated ricin A chain. J. Immun. Meth. 1988, 112:267.
53. Bjorn M. J., Ring D., Frankel A. Evaluation of monoclonal antibodies for the development of breast cancer immunotoxins. Cancer Res. 1985, 45:1214.
54. Vitetta E. S., Stone M., Amlot P., et al. Phase I immunotoxin trial in patients with B-cell lymphoma. Cancer Res. 1991, 51:4052.

What is claimed is:

1. A pharmaceutical composition for eliminating or reducing the number of unwanted CD3 and/or CD7 positive cells, said pharmaceutical composition consisting of:
first molecules directed against CD3; and
second molecules, distinct from said first molecules, said second molecules targeting CD7, wherein at least one of said first and said second molecules include a toxic moiety.

2. The pharmaceutical composition of claim 1, wherein said second molecules are antibodies.

3. The pharmaceutical composition of claim 1, wherein said toxic moiety is chemically linked to said first and second molecules.

4. The pharmaceutical composition of claim 1, wherein said first and second molecules are provided with toxic moieties, wherein said toxic moieties are the same or different.

5. The pharmaceutical composition of claim 1, wherein said second molecules include a toxic moiety.

6. The pharmaceutical composition of claim 1, wherein said toxic moiety is ricin.

7. The pharmaceutical composition of claim 6, wherein said ricin is deglycosylated ricin A.

8. The pharmaceutical composition of claim 6, wherein the toxic moiety is at least the equivalent dose of 25 micrograms of ricin A per square meter of body surface of a subject to be treated with said pharmaceutical composition.

9. The pharmaceutical composition of claim 8, wherein the toxic moiety is at least the equivalent dose of 100 micrograms of ricin A per square meter of the subject's body surface per administration.

10. The pharmaceutical composition of claim 8, wherein the toxic moiety is at most the equivalent dose of 25 mg of ricin A per square meter of the subject's body surface per infusion.

11. The pharmaceutical composition of claim 1, wherein said first molecules are antibodies.

12. The pharmaceutical composition of claim 11, wherein said second molecules are antibodies.

13. The pharmaceutical composition of claim 11, wherein said first molecules are gamma2B IgG.

14. The pharmaceutical composition of claim 12, wherein said toxic moiety is deglycosylated ricin A.

15. The pharmaceutical composition of claim 12, wherein said toxic moiety is chemically linked to said first and second molecules.

16. The pharmaceutical composition of claim 15, wherein both said first and second molecules are provided with toxic moieties, which may be the same or different toxic moieties.

17. A method of treating a disease state in a subject believed to be suffering therefrom, said disease state selected from at least one of the group consisting of: Graft vs. Host disease, graft rejections, T-cell leukemias, T-cell lymphomas, other lymphomas, other CD3 and/or CD7 malignancies, autoimmune diseases, and infectious immune disease, said method comprising administering to the subject an amount of a pharmaceutical composition consisting of:
first molecules directed against a CD3 positive cell; and
second molecules, distinct from said first molecules, directed against a CD7 positive cell, wherein at least the second molecules include a toxic moiety.

18. The method according to claim 17, wherein said second molecules specifically recognize CD7.

19. A pharmaceutical composition for eliminating or reducing the number of unwanted CD3 and/or CD7 positive cells, said pharmaceutical composition consisting of:
anti-CD3 antibodies; and
anti-CD7 antibodies, wherein each of said anti-CD3 antibodies and said anti-CD7 antibodies include a toxic moiety.

20. A method of treating a disease state in a subject believed to be suffering therefrom, said disease state selected from at least of the group consisting of: Graft vs. Host disease, graft rejections, T-cell leukemias, T-cell lymphomas, other lymphomas, other CD3 and/or CD7 malignancies, autoimmune diseases, and infectious immune diseases, said method comprising administering to the subject an amount of a pharmaceutical composition consisting of:
anti-CD3 antibodies; and
anti-CD7 antibodies, wherein each of said anti-CD3 antibodies and said anti-CD7 antibodies include a toxic moiety.
